(19) United States
(12) Patent Application Publication
(10) Pub. No.: US 2020/0102547 A1
(43) Pub. Date: Apr. 2, 2020

(54) REPROSOMES, AS EXOSOMES CAPABLE OF INDUCING REPROGRAMMING OF CELLS AND PREPARATION METHOD THEREOF

(71) Applicant: STEMON Inc., Seongnam-si (KR)

(72) Inventor: Yong Seung LEE, Gyeonggi-do (KR)

(73) Assignee: STEMON Inc., Seongnam-si (KR)

(21) Appl. No.: 16/230,242

(22) Filed: Dec. 21, 2018

(30) Foreign Application Priority Data
Oct. 2, 2018 (KR) 10-2018-0117261

(51) Int. Cl. C12N 9/12; C12N 5/079; C12N 5/077; C12N 5/071

(52) U.S. Cl. CPC C12N 9/12; C12Y 207/11024; C12N 5/0618; C12N 2506/1307; C12N 5/067; C12N 5/0627; C12N 5/0653

(57) ABSTRACT

The present disclosure provides a reprosome that can induce reprogramming of a cell, in which the reprosome is characterized by including RNA of a gene involved in chromatin remodeling, in which the gene includes a kinase gene on a mitogen-activated protein kinase (MAPK) signal transduction system, and a gene having histone modification activity. The reprosome may be obtained from a stem cell difficult to process and/or from a readily obtainable somatic cell via a simple process including ultrasonic treatment. A reprogramming of one kind of a cell into another kind of a cell with a desired function can be achieved at a high efficiency in a short time via a simple treatment including a co-culturing between the reprosome and the cell. The cell thus obtained has the desired function without introduction of a chemical or foreign transcription factor into a genome and thus is more suitable for cell replacement therapies. Further, the present disclosure provides a composition including the reprosome and a method for regenerating a tissue by treating a body site with the composition to promote reprogramming of a cell present in the treated body site to a target cell having a desired function.

### BACKGROUND OF THE INVENTION

### Field of the Invention

The present invention relates to a reprosome as an exosome capable of inducing reprogramming of a cell, and a method for producing the same. More specifically, the present invention relates to a reprosome containing a large amount of a chromatin remodeling factor, a method capable of producing the reprosome with a high yield, and a use of the reprosome produced by the method.

### Description of the Related Art

An exosome is a nano-vesicle with a diameter of 30 to 200 nm that is secreted naturally. It is known that the exosome can act as an important nano-mediator carrying a genetic material from a derived cell. Since the discovery that the exosome alters a phenotype in a target cell via mRNA delivery, several studies have shown that the exosomes are involved in cell differentiation. In this conventional study, stem cells and precursor cells were used as cells used for obtaining the exosome or for inducing differentiation or phenotypic changes using the exosome. Separation and amplification of these cells require a careful process. As a result, in such an approach, there are problems in terms of efficiency and economy. If secretion of exosomes containing factors capable of changing cells in a desired direction can be induced from easily obtainable somatic sells of a patient, and somatic cells that can be easily obtained can be replaced with other cells having desired functions using such exosome, it will lead to a great leap for clinical application of a cell replacement therapy.

In addition, a possibility of using the exosome itself directly in the human body for therapeutic purposes is being sought in various ways. In the field of biologics, the exosome is at an intermediate position between biopharmaceuticals such as small molecules, peptides, growth factors, antibodies, and nucleic acids, and large drugs such as various cells and platelets. In other words, compared to biopharmaceuticals, exosomes contain a variety of proteins and nucleic acids, which can be more complex and lasting. Further, exosomes have other advantages compared to cells. That is, when a cell is injected into a living body, for example, there is a concern about safety in that the injected cell may change into a cancer cell. Further, it is known that cells injected into a blood vessel for cell therapy are generally filtered and trapped in an organ having a filtering function such as lung, liver, spleen or kidney, and the cells that are filtered as described above are eventually degraded into a small vesicle form and affect the human body. Therefore, when the exosome having a small size and containing an active ingredient capable of being secreted from the cells is administered, spatiotemporal treatment effects that are different from those of complete cells may be made safer.

However, the exosome having a high possibility to be used as a tool for cell therapy or as a therapeutic agent itself, as described above, is currently difficult to produce in large quantities. For example, the exosome that can be obtained from 60 million mesenchymal stem cells cultured in a 1-liter medium is about 1 to 2 mg based on the contained protein content. This is an amount that can be used for treatment trials of several mice. In humans, exosomes used for the treatment of, for example, Graft-versus-host disease (GVHD) are known to require from 0.05 to 0.6 mg per 1 kg patient body weight on a protein content basis in a single time treatment. Therefore, in order to use the exosome clinically, a technique for increasing the yield thereof is required.

The present invention meets a variety of needs, including the aforementioned needs existing in cell therapy and exosome therapeutics. In other words, stem cells commonly used for cell therapy has problems that they are difficult to separate, amplify and maintain, that they are difficult to predict the direction of the differentiation of the stem cells, and that they are likely to develop into cancer. In addition, the process of obtaining a cell with the desired function requires different stages of differentiation and takes longer, its conversion efficiency is low, and the cell is produced by chemical process, making it difficult to secure safety. Therefore, there is a need for a technique that makes it possible to use an easily obtainable cell for cell treatment and a technique for safely and efficiently obtaining a cell having a desired function within a short time. In the case of exosome treatment, there is a lack of technology to obtain a sufficient amount for treatment.

### SUMMARY OF THE INVENTION

The present invention is intended to solve the above problem. One embodiment of the present invention provides a reprosome that can induce reprogramming of a cell in a desired direction, in which the reprosome is characterized by including RNA of a gene involved in chromatin remodeling, in which the gene includes a kinase gene on a mitogen-activated protein kinase (MAPK) signal transduction system, and a gene having histone modification activity.

Further, one embodiment of the present invention provides a method for producing a large amount of the above-described reprosome in a short time, in which the method includes applying ultrasonic stimulation to a cell, applying ultrasonic stimulation to a culture medium free of a cell, and mixing the cell with the culture medium to culture the cell for a predetermined period of time.

Further, one embodiment of the present invention provides a method of effectively reprogramming a cell in a desired direction by administering the reprosome to the cell.

Further, an embodiment of the present invention provides a composition including the reprosome inducing reprogramming of a cell.

The technical purpose to be achieved by the present invention is not limited to the above-mentioned technical purpose. Other technical purposes not mentioned may be clearly understood by those skilled in the art to which the present invention belongs from following descriptions.

Technical solutions for achieving the above technical purpose are as follows. In one aspect of the present invention, there is provided a reprosome that can induce reprogramming of a cell in a desired direction, in which the reprosome is characterized by including RNA of a gene involved in chromatin remodeling, in which the gene includes a kinase gene on a mitogen-activated protein kinase (MAPK) signal transduction system, and a gene having histone modification activity.

In this connection, the kinase gene on the MAPK signal transduction system may be at least one selected from a group consisting of BRAF, MAP2K3, MAP3K10, MAP3K4, MAP3K5, MAP3K7, MAPK12, RPS6KA4(MSK2), TAOK1, and TAOK2.

The gene having the histone modification activity may be at least one selected from a group consisting of ASH1L, CREBBP, DOT1L, EP300, GTF3C1, KAT2A, KAT6B, KDM1A, KDM3B, KDM6A, KMT2A, KMT2E, NCOA3, NSD1, SETD1A, and SETD2.

The percentage of small RNAs in a total RNA in the reprosome is 40% or more, and the percentage of microRNA (miRNA) in the small RNA is 40% or more.

In another aspect of the present invention, there is provided a method for producing reprosomes that induce cell reprogramming, the method including applying ultrasonic stimulation to the cell; applying ultrasonic stimulation to a culture medium free of cells; mixing the cell and the culture medium to culture the cell for a predetermined time; and separating the reprosome from the mixture.

In this connection, the cells may be a mammalian-derived fibroblast or a tissue cell in an organ.

The culture medium includes at least one selected from a group consisting of an embryonic stem cell medium, a neural stem cell medium, a cardiac stem cell medium, a dermal papilla cell medium, a mesenchymal stem cell medium, an osteogenic medium, a muscular formation medium, a hematopoietic stem cell medium, a neuron medium, an astrocyte medium, an oligodendrocyte medium, a hepatocyte medium, an adipocyte medium, a muscle cell medium, a vascular endothelial cell medium, a pancreatic beta cell medium, and a cardiac myocyte medium.

The culture medium may be any one selected from a group consisting of a neural stem cell medium, a dermal papilla cell medium, a hepatocyte medium, and an adipocyte medium.

The ultrasonic stimulation applied to the cells may be performed for 1 to 10 seconds at 10 to 30 kHz, and 0.5 to 3 W/cm².

The ultrasonic stimulation applied to the culture medium may be performed for 1 to 20 minutes at 10 to 30 kHz, 1 to 20 W/cm².

The culturing of the mixture may be characterized as proceeding for 1 to 10 days.

The step of separating the reprosome may include centrifuging the mixture after the culturing and obtaining a supernatant; filtering the supernatant with a filter and obtaining a filtrate; and concentrating the filtrate. In this connection, the step of separating the reprosome may further include storing the supernatant at 4° C. or below for 7 days to 1 month before filtering the supernatant with the filter. The separated reprosome has a diameter of 50 to 200 nm.

In still another aspect of the present invention, there is provided a method for reprogramming a cell, the method including introducing the reprosome into a first culture medium to form a mixture, culturing a first cell in the mixture; and obtaining a second cell after the culturing.

In this connection, the first cell may be a mammalian-derived fibroblast or a tissue cell in an organ.

The second cell may be a cell having differentiation ability equal to or lower than pluripotency.

The second cell may be any one selected from a group consisting of embryonic stem cells, neural stem cells, cardiac stem cells, dermal papilla cells, mesenchymal stem cells, and hematopoietic stem cells.

The second cell can be any one selected from a group consisting of neural stem cells, neurons, astrocytes, oligodendrocytes, hepatocytes, adipocytes, hair follicle cells, muscle cells, vascular endothelial cells, keratinocytes, pancreatic beta cells, and cardiac myocytes.

The second cell may be characterized as being of a type different from the first cell.

The reprosome may be characterized by being introduced into the first culture medium at a concentration of 10⁷ to 10¹⁵ cells/ml.

The first culture medium may be characterized by being the same culture medium as the culture medium used to produce the reprosomes.

The second cell may be either a stem cell, a progenitor cell or precursor cell. The first cell culturing may be characterized as being performed for 1 to 6 days.

The second cell may be any one selected from a group consisting of a neuron, an astrocyte, an oligodendrocyte, a hepatocyte, an adipocyte, a hair follicle cell, a muscle cell, a vascular endothelial cell, a keratinocyte, a pancreatic beta cell, and a cardiac myocyte. The first cell culturing may be characterized by progressing for 10 days to 60 days.

In still another aspect of the present invention, there is provided a composition that includes the reprosomes that induce reprogramming of cells, as described above.

### BRIEF DESCRIPTION OF THE DRAWINGS

The application file contains at least one drawing executed in color. Copies of this patent or patent application publication with the color drawing(s) will be provided by the Office upon request and payment of the necessary fee.

FIG. 1 illustrates generation data of reprosome with neural precursor cell (NPC) inducing ability induced according to Example 1 of the present invention: electron microscope images (A) illustrating the morphology of exosomes (iExo (reprosome) and nExo) secreted from ultrasound treated cells (UHDF) and non-ultrasound treated cells (NHDF); immunofluorescent staining image (B) illustrating inducement of CD63; graphs (C and D) illustrating the size distribution and number of the exosomes; graph (E) illustrating the change of exosome secretion amount over time after the start of culturing.

FIG. 2 illustrates components data of the reprosome with the neural precursor cell (NPC) inducing ability induced according to Example 1 of the present invention: (A) exosome RNA levels, (B) number of genes expressed with the RNA, (C) amount of small RNAs and miRNAs in the RNA, (D) concentration of protein in the exosome, (E and F) relative expression levels of NPC marker mRNA (Sox1, Sox2, Pax6 and Nestin) and miRNA (miR9, miR124a, miR125b and miR128-1) in the exosome, (G) heat-map illustrating neural associated mRNA (left) and miRNA expression (right), and (H) immunofluorescent staining image illustrating intracellular common distribution of NPC markers (Sox1, Sox2, Pax6 and Nestin) and exosome marker (CD63).

FIG. 3 illustrates analysis data of the NPC inducing ability of reprosomes with neural precursor cell (NPC) inducing ability isolated according to Example 1 of the present invention: (A) is a schematic diagram of a protocol that induces the cell, (B) is a confocal microscopy image illustrating the inflow of reprosomes into the cells over time after reprosome (iExo) treatment, (C) illustrates the form change of HDF, (D) illustrates qRT-PCR data for NPC markers (Sox1, Sox2, Pax6 and Nestin) RNAs, (E) and (F) illustrate immunofluorescent staining images of the marker proteins and flow cytometry data for the marker proteins, (G) illustrates cluster analysis data for NSC-related genes, (H) illustrates an immunofluorescent staining image for a cell division marker (Ki-67), (I) and (J) illustrate the number of spheroids formed by the exosome treatment concentration and expression levels of the NPC markers (Sox1, Sox2, Pax6 and Nestin), and (K) illustrates a growth curve of a 6th cell of subculture.

FIG. 4 illustrates analysis data of induction mechanism of the neural precursor cell (rNPC) according to Example 1 of the present invention: (A) illustrates a heat-map of chromatin remodeling-related gene expression in the nExo and reprosome (iExo)-treated cell groups, (B) and (C) illustrate Western blot analysis illustrating the degree of phosphorylation of MAPK signal transduction system proteins (Erk1/2, p38 and Msk1) over time after the reprosome treatment, and intracellular immunofluorescence staining image, (D) illustrates qRT-PCR data for NPC markers (Sox1, Sox2, Pax6 and Nestin), (E) illustrates immunofluorescent staining data for chromatin remodeling index (HP1α, H3K4me3, H3K27me3) and distribution of the indices relative to the contrast staining DAPI, (F) illustrates a heat-map of a neural-related gene expression in each cell group.

FIG. 5 illustrates analysis data of in vitro and in vivo differentiation ability of rNPC according to Example 1 of the present invention: (A) illustrates immunofluorescent staining data of neuronal markers (neuron marker Map2 and Tuj1, astrocyte marker Gfap, oligodendrocyte marker 04) for rNPC which is cultured for 4 weeks and differentiated in a neural differentiation medium, (B) and (C) illustrate in vitro differentiation ability data, as indicated by voltage clamp data and action potential data for the differentiated rNPCs, (D) illustrates a distribution of GFP-rNPC after 4 weeks of transplantation in rat brain, (E) illustrates immunofluorescence staining results using human mitochondrial antibody and GFP, (F) illustrates immunofluorescent staining results for the neural markers.

FIG. 6 illustrates production data of reprosomes with adipocyte inducing ability according to Example 2 of the present invention: (A) illustrates nanosight and electron microscope images confirming the shape of iExo (reprosome), (B) and (C) illustrate an immunofluorescent staining image illustrating inducing of exosome marker CD63 and coexistence between the CD63 and brown adipocyte marker UCP1, and (D) illustrates RNA-seq data for characteristics analysis of brown adipocyte-associated mRNA/miRNA and lipid synthesis-related mRNA in isolated reprosome.

FIG. 7 illustrates immunofluorescence staining data of brown adipocyte (rBA) according to Example 2 of the present invention: This is an immunofluorescent staining image using lipid staining reagent AdipoRed, human mitochondrial antibody (HuMito), and antibody against brown adipocyte marker UCP1.

FIG. 8 illustrates production data of reprosomes with hepatocyte inducing ability according to Example 3 of the present invention: (A) illustrates nanosight and electron microscope images confirming the shape of iExo (reprosome), (B) and (C) illustrate immunofluorescent staining images illustrating inducing of exosome marker CD63, and coexistence between the CD63 and hepatocyte marker HNF1α, and (D) illustrates RNA-seq data for characteristics analysis of hepatocyte-associated mRNA/miRNA in isolated reprosome.

FIG. 9 illustrates immunofluorescence staining data of hepatocyte (rH) according to Example 3 of the present invention: this is an immunofluorescent staining image for hepatocyte markers AFP, HNF4α, CK18 and ALB.

FIG. 10 illustrates production data of reprosomes with the hair tissue differentiation-inducing ability according to Example 4 of the present invention: (A) illustrates nanosight and electron microscope images confirming the shape of iExo (reprosome), (B) and (C) illustrate immunofluorescent staining images illustrating inducing of CD63 as an exosome marker, and coexistence between CD63 and Shh, as an important protein for hair regeneration, and (D) illustrates RNA-seq data of characteristics analysis of mRNAs associated with hair regeneration in isolated reprosome.

FIG. 11 illustrates data of in vivo gene expression changes in C57 and nude mouse skin by reprosomes with hair regeneration ability according to Example 4 of the present invention: (A) and (B) illustrate fluorescence staining images illustrating a post-application distribution after application reprosomes labeled with lipophilic marker Did, (C) and (D) illustrate immunofluorescent staining images of proteins (β-Catenin, Shh, Ki-67) involved in the expression of the hair follicle regeneration gene by the reprosome treatment, and (E) and (F) illustrate qRT-PCR data for mRNA (Shh, beta-Catenin, KRT-25, VCAN, Gli1, Lef1, Pct1, Tyrp1, Tyr, Mitf, Dct, Sfrrp4, DKK).

FIG. 12 illustrates tissue changes data in C57 and nude mouse skin by reprosome with hair regeneration ability according to Example 4 of the present invention: H&E staining images of skin tissue (A and B) and follicular number data in subcutaneous and whole skin (C and D).

FIG. 13 illustrates hair regeneration data in C57 and nude mouse skin by reprosome with hair regeneration ability according to Example 4 of the present invention: (A) and (B) illustrate hair generation images based on exosome treatment concentration and over time after exosome treatment.

FIG. 14 illustrates production data of the reprosome with wound-healing ability according to Example 5 of the present invention: (A) illustrates immunofluorescent staining images illustrating the inducing of CD63 as an exosome marker, (B) illustrates qRT-PCR data of analysis of RNA of wound-healing genes in induced reprosome, (C) illustrates RNA-seq data.

FIG. 15 illustrates in vitro effect data by reprosomes with wound-healing ability according to Example 5 of the present invention: (A) illustrates immunofluorescent staining images illustrating expression of cell proliferation marker Ki67 in HDF treated with various concentrations of the reprosome, (B) illustrates time-dependent proliferation rate of HDF based on different treatment concentrations of reprosome as measured by MTT assay, (C) and (D) illustrate time-dependent migration ability of HDF based on different treatment concentrations of the reprosome as measured by scratch assay, (E) to (G) illustrate optical microscope images illustrating tube formation of HUVEC based on various reprosome treatment concentrations, the length of the formed tube and number of crossing points, and (H) illustrates qRT-PCR data of analyzing RNA of intracellular wound-healing gene treated with reprosome.

### DETAILED DESCRIPTION OF THE PREFERRED EMBODIMENTS

Examples of various embodiments are illustrated and described further below. It will be understood that the description herein is not intended to limit the claims to the specific embodiments described. On the contrary, it is intended to cover alternatives, modifications, and equivalents as may be included within the spirit and scope of the present disclosure as defined by the appended claims.

The terminology used herein is for the purpose of describing particular embodiments only and is not intended to be limiting of the present disclosure. As used herein, the singular forms “a” and “an” are intended to include the plural forms as well, unless the context clearly indicates otherwise. It will be further understood that the terms “comprises”, “comprising”, “includes”, and “including,” when used in this specification, specify the presence of the stated features, integers, operations, elements, and/or components, but do not preclude the presence or addition of one or more other features, integers, operations, elements, components, and/or portions thereof.

In one aspect of the present invention, there is provided a reprosome that can induce reprogramming of a cell in a desired direction, in which the reprosome is characterized by including RNA of a gene involved in chromatin remodeling, in which the gene includes a kinase gene on a mitogen-activated protein kinase (MAPK) signal transduction system, and a gene having histone modification activity.

In this connection, the kinase gene on the MAPK signal transduction system may be at least one selected from a group consisting of BRAF (B-Raf proto-oncogene), MAP2K3 (Mitogen-activated protein kinase kinase 3), MAP3K10 (Mitogen-activated protein kinase kinase kinase 10), MAP3K4 (Mitogen-activated protein kinase kinase kinase 4), MAP3K5 (Mitogen-activated protein kinase kinase kinase 5), MAP3K7 (Mitogen-activated protein kinase kinase kinase 7), MAPK12 (Mitogen-activated protein kinase 12), RPS6KA4 (Ribosomal protein S6 kinase A4, also known as Msk2), TAOK1 (TAO kinase 1) and TAOK2(TAO kinase 2). Preferably, the kinase gene may include at least MAP3K10, RPS6KA4, and TAOK1 among the 10 genes. More preferably, the kinase gene may include at least MAP2K3, MAP3K10, MAPKK7, MAPK12, RPS6KA4, TAOK1 and TAOK2 among the 10 genes.

The gene having the histone modification activity may be at least one selected from a group consisting of ASH1L (ASH1 like histone lysine methyltransferase), CREBBP(CREB binding protein), DOT1L (DOT1 like histone lysine methyltransferase), EP300 (E1A binding protein P300), GTF3C1 (General transcription factor IIIC subunit 1), KAT2A (Lysine acetyltransferase 2A), KAT6B (Lysine acetyltransferase 6B), KDM1A (Lysine demethylase 1A), KDM3B (Lysine demethylase 3B), KDM6A (Lysine demethylase 6A), KMT2A (Lysine methyltransferase 2A), KMT2E (Lysine methyltransferase 2E), NCOA3 (Nuclear receptor coactivator 3), NSD1 (Nuclear receptor binding SET domain protein 1), SETD1A (SET domain containing 1A) and SETD2 (SET domain containing 2). Preferably, the gene having the histone modification activity may include at least ASH1L, CREBBP, DOT1L, EP300, GTF3C1, KAT6B, KDM1A, KDM3B, KMT2A, KMT2E and NSD1 among the 16 genes. More preferably, the gene having the histone modification activity may include at least ASH1L, CREBBP, DOT1L, EP300, GTF3C1, KAT6B, KDM1A, KDM3B, KMT2A, KMT2E, NCOA3, NSD1 and SETD2 among the 16 genes.

A small RNA is an RNA with a size of smaller than 200 nt (nucleotides), and is mainly a non-coding RNA. The small RNA may include microRNAs, short interfering RNAs, short nucleolar RNAs, piwi-interacting RNAs (piRNAs), and the like. Among them, the miRNA has a length of about 22 nt and is known to be involved in RNA silencing and post-transcriptional gene regulation. The percentage of small RNA in a total RNA in the reprosome is 40% or more, and the percentage of miRNA in the small RNA is 40% or more. More preferably, the percentage of small RNA in the total RNA in the reprosome is 50% or more. and the percentage of miRNA in the small RNA is 50% or more. Most preferably, the percentage of small RNA in the total RNA in the reprosome is 60% or more, and the percentage of miRNA in the small RNA is 60% or more.

The reprosomes, which may induce reprogramming of cells as described above, may be characterized by being produced by the producing method described later.

In another aspect of the present invention, there is provided a method for producing reprosomes that induce cell reprogramming, the method including applying ultrasonic stimulation to the cell; applying ultrasonic stimulation to a culture medium free of cells; mixing the cell and the culture medium to culture the cell for a predetermined time; and separating the reprosome from the mixture.

In this connection, the cell may preferably be selected from cells other than germ cells, preferably it may be a mammalian-derived fibroblast or a tissue cell in an organ. This is because the reprosome can be obtained using any cell using the method for producing a reprosome inducing cell reprogramming according to one aspect of the present invention, it is more efficient and easier to obtain, maintain and amplify fibroblasts or tissue cells in the organ than stem cells or precursor cells, which are hard to obtain and are difficult to amplify. The cell may be either autologous, allogeneic or heterologous depending on the future use of the reprosome with respect to other cells or organisms as will be described below. When the cell is heterologous, the cell may be derived from a mammal. Since there is a possibility for an immune rejection reaction, preferably the cell may be allogeneic, most preferably autologous.

In the step of applying ultrasonic stimulation to the cells, the cells may be directly subjected to ultrasonic treatment, or the ultrasonic treatment may be carried out at a minimal amount of cells so that the cells are barely covered by an initial culture medium. The initial culture medium is a common medium used to maintain the cells in a healthy state and is a medium suitable for normal culturing of the cells. For example, the cell may be a fibroblast, and the medium may be a DMEM medium containing an antibiotic and serum.

The culture medium (the ultrasound-treated medium) may be any one selected from a group consisting of an embryonic stem cell medium, a neural stem cell medium, a cardiac stem cell medium, a dermal papilla cell medium, a mesenchymal stem cell medium, an osteogenic medium, a muscle forming medium, a hematopoietic stem cell medium, a neuron medium, an astrocyte medium, an oligodendrocyte medium, a hepatocyte medium, an adipocyte medium, a muscle cell medium, a vascular endothelial cell medium, a pancreatic beta cell medium, and a cardiac myocyte medium. The culture medium may be any medium with differentiation inducing or maintenance and amplification purposes. According to another aspect of the present invention described below, when a first cell is to be reprogrammed into a second cell via treating with the reprosome, the medium may be selected which preferably allows the second cell to be obtained to be maintained and amplified in a healthy manner. According to another aspect of the present invention described below, when the composition including the reprosome is administered to a body site to promote reprogramming of the cells in the body site to target cells, and preferably, the medium that may maintain and amplify the cells in a healthy manner when the target cells are cultured in vitro may be selected.

The culture medium may be any one selected from a group consisting of a neural stem cell medium, a dermal papilla cell medium, a hepatocyte medium, and an adipocyte medium.

The ultrasonic stimulation applied to the cells may be performed for 1 to 10 seconds at 10 to 30 kHz, and 0.5 to 3 W/cm². Preferably, the ultrasonic stimulation applied to the cells may be performed for 3 to 7 seconds at 15 to 25 kHz, and 0.5 to 1.5 W/cm².

The ultrasonic stimulation applied to the culture medium may be performed for 1 to 20 minutes at 10 to 30 kHz, 1 to 20 W/cm². Preferably, the ultrasonic stimulation applied to the culture medium may be performed for 7 to 13 minutes at 15 to 25 kHz, 0.5 to 1.5 W/cm².

The culturing of the mixture may be characterized as proceeding for 1 to 10 days. The culturing of the mixture may preferably be carried out for 1 to 6 days, most preferably for 1 to 2 days. This is because the reprosome is secreted at a maximum degree on the first day after the ultrasonic treatment and the amount of reprosome secretion decreases over time, and thus, there is a possibility that there is a change in components considering this decrease in the secretion amount.

The step of separating the reprosome may include centrifuging the mixture after the culturing and obtaining a supernatant; filtering the supernatant with a filter and obtaining a filtrate; and concentrating the filtrate. The centrifugation is carried out to remove cell debris and dead cells. Preferably, the centrifugation may be conducted at 1000 to 5000 g for 10 minutes to 60 minutes. The step of filtering the supernatant with a filter is performed to remove more cell debris and leave only particles of a certain size or smaller. In this connection, the filter used may preferably be a syringe filter. The step of concentrating the filtrate may preferably be carried out using a centrifugal filter. Using the centrifugal filters may concentrate the filtrate while simultaneously removing particles below a certain size. The step of separating the reprosome may further include storing the supernatant at 4° C. or below for 7 days to 3 months before filtering the supernatant with the filter. The storage may preferably be performed within 7 days at 4° C. or lower, more preferably within 1 month at −20° C. or lower. Most preferably, the storage may be performed within three months at −80° C. or lower. Active ingredients of reprosome are mRNA and protein. The higher the temperature is or the closer the temperature is to a temperature at which the enzyme activity is high, the more easily these active ingredients may be denatured or decomposed. The separated reprosome may be characterized by a diameter of 50 to 200 nm, and preferably a diameter of 100 to 150 nm.

In still another aspect of the present invention, there is provided a method for reprogramming a cell, the method including introducing the reprosome into a first culture medium to form a mixture, culturing a first cell in the mixture; and obtaining a second cell after the culturing.

In this connection, the first cell may be a mammalian-derived fibroblast or tissue cell in an organ. This is because the second cell can be obtained using any somatic cell when using any cell using the method for reprogramming the cell according to one aspect of the present invention, it is more efficient and easier to obtain, maintain and amplify fibroblasts or tissue cells in the organ than stem cells or precursor cells, which are hard to obtain and are difficult to amplify. If the second cell is later transplanted into the human body, the first cell may preferably employ a human-derived cell. Most preferably, an autologous cell derived from a subject to be transplanted may be employed as the first cell. When a cell derived from a living organism genetically close to the subject to be transplanted with the second cell is used as the first cell, this may lower the possibility of adverse reactions such as rejection of cell transplantation.

The second cell may be a cell having differentiation ability equal to or lower than pluripotency.

The second cell may be any one selected from a group consisting of embryonic stem cells, neural stem cells, cardiac stem cells, dermal papilla cells, mesenchymal stem cells, and hematopoietic stem cells.

The second cell can be any one selected from a group consisting of neural stem cells, neurons, astrocytes, oligodendrocytes, hepatocytes, adipocytes, hair follicle cells, muscle cells, vascular endothelial cells, keratinocytes, pancreatic beta cells, and cardiac myocytes. The differentiation ability of cells that have not been terminally differentiated may be classified as totipotency, pluripotency, multipotency, oligopotency, and unipotency when enumerated from a high level to a low level. Totipotency refers to the ability by which the cells that have not been terminally differentiated may be differentiated into all cells of one organism and that one organism may be formed from one cell. Pluripotency refers to the ability by which the cells that have not been terminally differentiated may be differentiated into all three endoderm, mesoderm and ectoderm cells. Multipotency refers to the ability by which the cells that have not been terminally differentiated may be differentiated into several cells of a lineage or a few lineages. Oligopotency refers to the ability by which the cells that have not been terminally differentiated may be differentiated into several cells of a range smaller than the range as described above with reference to the multipotency. Unipotency refers to the ability by which the cells that have not been terminally differentiated may be differentiated into one cell type. Thus, cells with differentiation ability equal to or below the pluripotency may include the pluripotency cell, multipotency cell, oligopotency cell, and unipotency cell and the terminally differentiated cell. If the second cell is transplanted into the human body in the future, the second cell is preferably a stem cell, a progenitor cell or precursor cell with multipotency, oligopotency or unipotency. The possibility that pluripotency cells may be transformed into cancer cells has been continuously raised, and the terminally differentiated cells may have a short lifespan or may be less effective for cell therapy.

The second cell may be characterized as being of a type different from the first cell. For example, the first cell may be fibroblasts that are easy to obtain and maintain, or cells obtained from other tissues. The method for reprogramming cells according to the present invention may easily obtain a desired second cell.

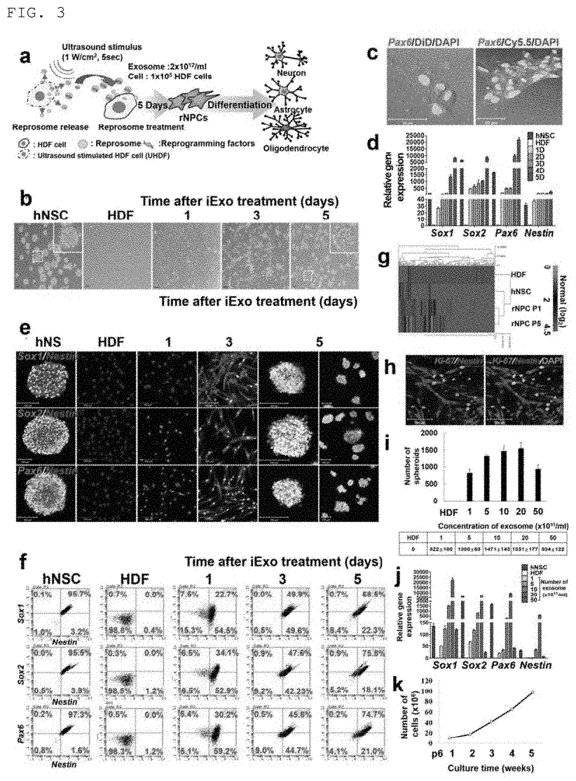

The reprosome may be characterized by being introduced into the first culture medium at a concentration of 10⁷ to 10¹⁵ cells/ml. More preferably, the reprosome may be characterized by being introduced into the first culture medium at a concentration of 10¹⁰ to 10¹² cells/ml. The present inventors could also confirm that the reprogramming efficiency is poor if the concentration of the introduced reprosome is too low or high (FIG. 3I).

The first culture medium may be characterized by being the same culture medium as the culture medium used to produce the reprosomes, but the present disclosure is not limited thereto. This is because the first cell may be reprogrammed more quickly into the desired second cell when the first culture medium is the same as the culture medium used to produce the reprosome, but the first cell can be reprogrammed into the second cell if reprosomes are introduced even when a different culture medium is used.

In one example, the second cell may be either stem cell, progenitor cell or precursor cell. The first cell culturing may be characterized as being performed for 1 to 6 days. In another example, the second cell may be any one selected from a group consisting of a neuron, astrocyte, an oligodendrocyte, a hepatocyte, an adipocyte, a hair follicle cell, a muscle cell, a vascular endothelial cell, a keratinocyte, a pancreatic beta cell and a cardiac myocyte. The first cell culturing may be characterized by progressing for 10 days to 60 days. Since the time required for reprogramming to cells having high differentiation ability and the time required for reprogramming to cells having low differentiation ability are different and the latter reprogramming tends to take more time, the latter case may preferably be cultured for 15 days to 25 days.

In still another aspect of the present invention, there is provided a composition that includes the reprosomes that induce reprogramming of cells, as described above. In this connection, the composition may be administered to a site of the body, for example, to promote the regeneration of tissues present in the site. The body site may include the epidermis, the dermis, and the scalp. In this case, the composition may be applied on or injected into the site, and thus may have a tissue regeneration effect, such as wound healing or hair regeneration.

It is obvious that the composition may contain pharmaceutically acceptable carriers and/or additives such as additives that may increase the permeability to the body site to be treated to further enhance the tissue regeneration effect, in addition to the reprosome. Descriptions of the pharmaceutically acceptable carriers and/or additives may be omitted.

The reprosomes according to one embodiment of the present invention contain a variety of reprogramming factors, particularly chromatin remodeling factors and has a phospholipid-based membrane structure, to allow induction of reprogramming into cells with desired functions with high efficiency.

The method for producing the reprosomes according to one embodiment of the present invention may induce a secretion of reprosome containing reprogramming factors for various cells from readily obtainable somatic cells as well as stem cells and progenitor cells, which are difficult to isolate and amplify, via a simple process including ultrasonic treatment.

The reprogramming method of cells according to one embodiment of the present invention can safely induce reprogramming into cells with the desired function without the introduction of chemicals or foreign transcription factors into the genome. Further, the reprogramming method can reprogram one type of cell into another type of cell in a relatively short period of time, without the need to go through several developmental stages, via simply adding the reprosome to the culture medium and culturing the cell.

The composition including the reprosome according to one embodiment of the present invention may be administered to a body site to promote tissue regeneration of the body site.

Hereinafter, Examples of the present invention will be described in detail so that a person skilled in the art can easily carry out the present invention. However, the present invention may be embodied in many different forms and may not be limited to Examples described herein.

All cell culturings on all Examples and Experiment Examples of the present invention were performed at 37° C. and 5% CO₂.

### Example 1. Producing Reprosomes with Neural Precursor Cell Inducing Ability and Inducing Reprogramming of Fibroblasts into Neural Precursor Cells

To obtain reprosomes with neural precursor cell inducing ability, first, ultrasonic stimulation at 20 kHz and 1.0 W/cm² was directly applied for 5 seconds to the 1×10⁶ HDFs using UltraRepro 1001 (STEMON Inc., Seoul, Republic of Korea) (Hereinafter, this stimulated HDF is referred to as UHDF). 2×10⁵ UHDFs and an ultrasound-treated (at 20 kHz and 5.0 W/cm², for 10 min) hNSC medium together were cultured in a 35-mm petri dish for one day. The reprosomes were isolated from the UHDF culture medium as follows: the culture medium was centrifuged at 3,000×g for 20 minutes to remove the cell debris and dead cells. Subsequently, a supernatant was passed through a 0.22-mm filter (Minisart® Syringe Filter, Sartorius, Goettingen, Germany). The filtered medium was placed in an Amicon® Ultra-15 100,000 kDa device (Millipore, Billerica, Mass., USA). Then, the medium was centrifuged at 14,000×g for 20 minutes such that the reprosome (iExo) was concentrated.

To produce rNPC (neural precursor cell obtained by reprogramming using reprosome), 1×10⁵ HDFs were seeded in a 35-mm petri dish and cultured for one day. Then, an existing medium was replaced with an hNSC medium containing the reprosomes isolated from the medium and then, culturing was conducted for 5 days. The culture medium was replaced every two days.

### Example 2. Producing Reprosomes with Adipocyte Inducing Ability and Inducing Reprogramming of Fibroblasts into Brown Adipocyte

To obtain reprosomes with adipocyte inducing ability, first, ultrasonic stimulation at 20 kHz and 1.0 W/cm² was directly applied for 5 seconds to the 1×10⁶ HDFs using UltraRepro 1001 (STEMON Inc., Seoul, Republic of Korea). 2×10⁵ UHDFs and an ultrasound-treated (at 20 KHz and 5.0 W/cm², for 10 min) adipocyte differentiation-inducing medium for stem cell (MesenCult™ adipogenetic Differentiation Medium, Stemcell technologies) together were cultured in a 35-mm petri dish for one day. The reprosomes were isolated from the UHDF culture medium as in Example 1.

To produce rBA (brown adipocyte obtained by reprogramming using reprosome), 1×10⁵ HDFs were seeded in a 35-mm petri dish and cultured for one day. Then, an existing medium was replaced with an adipocyte differentiation-inducing medium including the reprosomes isolated from the medium and then, culturing was conducted for 20 days. The culture medium was replaced every two days.

### Example 3. Producing Reprosomes with Hepatocyte Inducing Ability and Inducing Reprogramming of Fibroblasts into Hepatocyte

To obtain reprosomes with hepatocyte inducing ability, first, ultrasonic stimulation at 20 kHz and 1.0 W/cm² was directly applied for 5 seconds to the 1×10⁶ HDFs using UltraRepro 1001 (STEMON Inc., Seoul, Republic of Korea). 2×10⁵ UHDFs and an ultrasound-treated (at 20 kHz and 5.0 W/cm², for 10 min) hepatocyte culture medium (HCM™ hepatocyte culture medium, Lonza) together were cultured in a 35-mm petri dish for one day. The reprosomes were isolated from the UHDF culture medium as in Example 1.

To produce rH (hepatocyte obtained by reprogramming using reprosome), 1×10⁵ HDFs were seeded in a 35-mm petri dish and cultured for one day. Then, an existing medium was replaced with the hepatocyte-culture medium including the reprosomes isolated from the medium and then, culturing was conducted for 24 days. The culture medium was replaced every two days.

### Example 4. Producing Reprosomes with Hair Regeneration Ability and Inducing Hair Regeneration Using the Reprosomes

To obtain reprosomes with hair regeneration ability, first, ultrasonic stimulation at 20 kHz and 1.0 W/cm² was directly applied for 5 seconds to the 1×10⁶ HDFs using UltraRepro 1001 (STEMON Inc., Seoul, Republic of Korea). 2×10⁵ UHDFs and an ultrasound-treated (at 20 KHz and 5.0 W/cm², for 10 min) dermal papilla cell culture medium (Promocell) together were cultured in a 35-mm petri dish for one day. The reprosomes were isolated from the UHDF culture medium as in Example 1.

### Example 5. Producing Reprosomes with Wound Healing Ability

To obtain reprosomes with wound healing ability, first, ultrasonic stimulation at 20 kHz and 1.0 W/cm² was directly applied for 5 seconds to the 1×10⁶ HDFs using UltraRepro 1001 (STEMON Inc., Seoul, Republic of Korea). 2×10⁵ UHDFs and an ultrasound-treated (at 20 kHz and 5.0 W/cm², for 10 min) embryo stem cell culture medium (DMEM/F12, 15% FBS, 2 mM GlutaMAX, 0.1% NEAA, 0.1% penicillin/streptomycin, 0.1 mM β-mercaptoethanol, 1000 unit/ml leukemia inhibitory factor (LIF)) together were cultured in a 35-mm petri dish for one day. The reprosomes were isolated from the UHDF culture medium as in Example 1.

### Experiment Example 1. Experiment to Produce Reprosomes with Neural Precursor Cell Inducing Ability

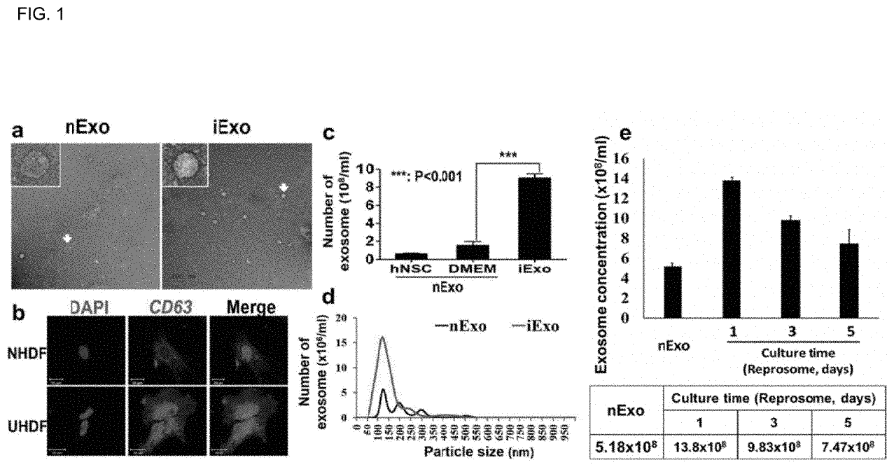

To induce the secretion of reprosomes with neural precursor cell inducing ability, HDFs were exposed to ultrasonic stimulation to generate UHDFs according to Example 1, and cultured in a human neural stem cell medium (hNSC medium) treated with ultrasound for one day. After the culturing for one day, it was confirmed using an exosome-specific marker CD63 (FIG. 1B) that a larger amount of vesicles was induced from the UHDF compared to NHDF (non-ultrasound treated HDF). The exosome (iExo) induced from the medium that cultured UHDF and the exosome (nExo) secreted from the NHDF were separated from each other using the reprosome separation method in Example 1. Transmission electron microscopy images showed that both the former and the latter had a common vesicular structure of the exosome (FIG. 1A). From the results of the nanoparticle tracing analysis, the particle size of iExo was in the range of about 50 to 200 nm, and the mean particle size thereof was 155.6±4.2 nm (FIG. 1C). The number of iExo particles of 50 to 200 nm is 9×10⁸. This value was 3.2 times higher than that of nExo particles (FIG. 1D). This result showed that ultrasound can be applied to the HDF to induce the exosome efficiently. The secretion amount of iExo was the highest on the first day based on the culturing point after the ultrasonic treatment. Although the secretion amount tended to decrease over time, the inventors could confirm that all of secretions at 1, 3, and 5 days after the culturing point were higher than those of nExo (FIG. 1E).

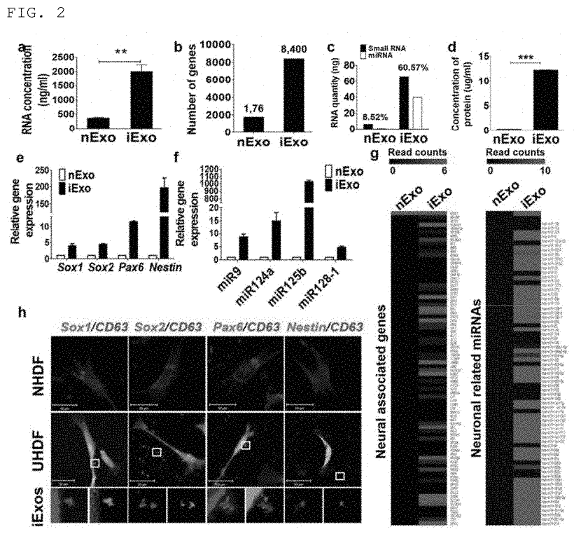

To analyze components of iExo, total RNAs and microRNA (miRNA) concentrations and qualitative parts from exosomes isolated from a first day-culture medium were measured using Agilent 2100 Bioanalyzer and the number of protein-coding genes was measured via exosome RNA sequencing (RNA-Seq). The total RNA concentration in iExo was 5.3 times higher than that of nExo (FIG. 2A), and the number of genes expressed into the RNA in iExo was 8400, which was 4.7 times higher than the RNA in nExo, which was 1762 (FIG. 2B). An open database search and gene ontology analysis revealed that some of the mRNAs in iExo were associated with neural development (FIG. 2G). The qRT-PCR (quantitative reverse transcription polymerase chain reaction) revealed that the expression level of NPC-specific marker genes such as Sox1, Sox2, Pax6 and Nestin in iExo was increased (FIG. 2E). The percentage of miRNA of iExo (60.57%) among non-coding small RNAs in the exosome was higher compared to that of nExo (8.52%) (FIG. 2C). Further, 53 miRNAs out of 72 miRNAs in iExo were found to belong to a neural system-specific miRNA according to Parsons et al. (FIG. 2G). A total protein concentration of iExo was increased by 20 times compared to that of nExo (FIG. 1D). Immunofluorescent staining and flow cytometry analysis showed that iExo contained NPC-specific marker proteins including Sox1, Sox2, Pax6 and Nestin. Further, the immunofluorescent staining and flow cytometry analysis showed that an expression of the genes into a protein increased with mRNA expression (FIG. 1H). The results demonstrated that UHDF could be induced to secrete reprosomes richly containing mRNA, miRNA and protein associated with neural development. The number of transcripts and proteins in the reprosome induced from UHDF was dramatically increased, which means that as many of these genes play a role in maintaining the ability to differentiate in stem cells, this increase means that they may act as a critical factor in cell reprogramming.

### Experiment Example 2. Analysis Experiment of Neuronal Precursor Cell Inducing Ability of Reprosome According to Example 1

To determine whether cell reprogramming in the direction of the neural phenotype may be induced using the reprosome, the present inventors have checked whether iExo is delivered into the HDF. The present inventors labeled iExo with lipophilic tracer DiD (DiD-labeled iExo) and cultured the HDFs for one day. It was confirmed that the DiD-labeled iExo could be identified in the cytoplasm of HDF treated with iExo (FIG. 3C, left panel). Additionally, to ensure that the reprogramming factor inside the iExo is delivered into the HDF, the present inventors labeled the intact HDF with Cy5.5-labeled poly(A)27, and had an exosome (Cy5.5-exo) including poly(A)27-Cy5.5 induced by going through the production process of UHDF. HDF was treated with iExo containing the Cy5.5-exo and cultured for one day. The Cy5.5-exo was observed in the cytoplasm of HDF (FIG. 3C, right panel). Interestingly, increased expression of Pax6 appeared from the HDF treated with iExo at 24 hours after the treatment, which means that the reprosome is transferred into the HDF with high efficiency.

Next, the inventors checked whether iExo could induce cell reprogramming. HDF (1×10⁵, seventh subculture) was exposed to exosomes (hNSC-Exo, nExo, iExo, respectively) isolated from a culture medium in which hNSC, NHDF and UHDF were cultured for one day. Interestingly, when the three exosomes and HDF were co-cultured for five days, only iExo-treated cells (iExo-HDF) formed dense colonies (>100 μm in diameter) (FIG. 3B), and showed increase of NPC-specific markers from the first day after culturing (FIGS. 3D to 3F). When cells were treated with 20×10¹¹ iExos/ml, a level of spheroid formation and NPC-specific marker expression level were the highest for the iExo-HDF (FIG. 3I). Hereinafter, the effect of iExo was confirmed using the above concentration. On the 5th day after exosome treatment, the number of iExo-HDF colonies reached 1,500, and the expression levels of NPC-specific genes such as Sox1, Sox2, Pax6 and Nestin and proteins increased to levels comparable to those in hNSC to show NPC-like properties (FIGS. 3D and 3E). Finally, from the results of double-positive flow cytometry, it was confirmed that ratios of Pax6/Nestin, Sox1/Nestin and Sox2/Nestin in the iExo-HDF reached 74.7%, 68.5% and 75.8%, respectively (FIG. 3F). From the result of RNA-seq analysis, it was confirmed that the neural-specific gene expression profile of the iExo-HDF was similar to that of hNSC and was different from that of HDF (FIG. 3G). Gene ontology analysis confirmed that multiple genes overexpressed from the iExo-HDF were associated with neural development. This result demonstrated that the iExo induced cell reprogramming rapidly, thereby producing NPC-like cells from HDF in 5 days in a high yield. The iExo-HDF having characteristics similar to NPC on the fifth day was named rNPC, and dense colonies of iExo-HDF were named as rNPC first subculture group (p1).

The present inventors collected spheroids of rNPC p1 and sub-cultured several times to obtain a uniform cell group. From the result of the NPC-specific marker gene and protein analysis, it was confirmed that all of rNPCs of p2, p4, p6 and p10 expressed high levels of Sox1, Sox2, Pax6 and Nestin. Ki-67 immunofluorescence staining showed that rNPC was actively proliferating (FIG. 3H). The proliferating capacity of P6 rNPC was maintained for several weeks (FIG. 3K). The results suggest that the cell is a cell of a homogeneous and amplifiable cell group that maintains Ki67 and NPC marker expression while undergoing multiple subculturings. This means that rNPC possesses the amplification and magnetic regeneration ability of the NPC.

### Experiment Example 3. Analysis Experiment of rNPC Induction Mechanism

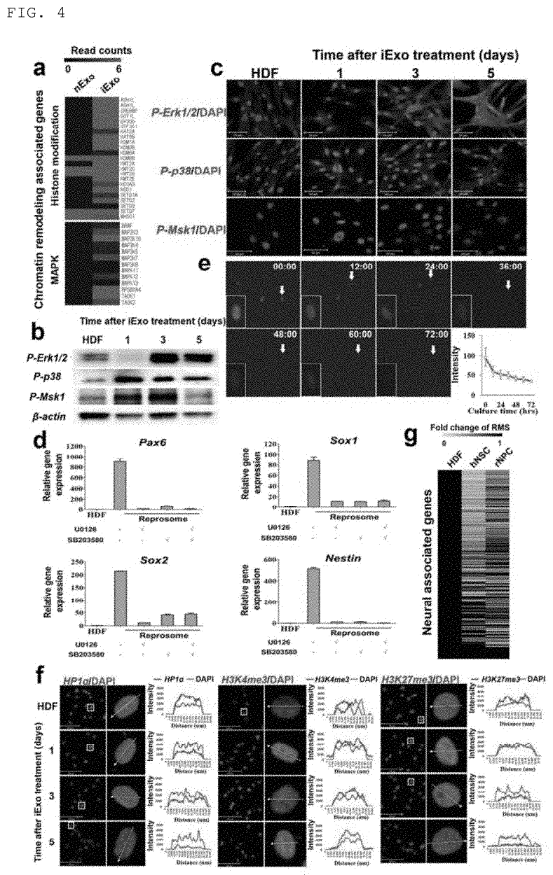

To elucidate the mechanism by which rapid cell reprogramming by reprosomes occurs, the present inventors have focused on the epigenetic regulation of gene expression by cell stress. Interestingly, iExo was including mRNA and protein associated with histone modification and mitogen-activated protein kinase (MAPK) pathway-related genes (FIG. 4A). Next, the present inventors checked if iExo simulates MAPL signal transduction pathway of the target cell, chromatin remodeling can be induced. Western blot and flow cytometry analysis showed that the expression levels of p38, Erk and Msk1 increased sharply from the iExo-treated HDF on a first day after the treatment (FIGS. 4B and 4C). Further, when iExo-HDF was treated with inhibitors 5B203580 and/or U0126 against p38 and Erk in the MAPK signal transduction pathway, the expressions of each target protein and NPC-specific genes and proteins such as Msk1 and Sox1, Sox2, Pax6 and Nestin, which are downstream proteins of the signal transduction pathway, may be confirmed to be significantly reduced (FIG. 4D). This result indicates that the MAPK signal transduction system acts as an important mechanism when the reprosome rapidly induces the rNPC via chromatin remodeling. In addition, at 3 days after iExo treatment, changes in local chromatin density and histone modification were observed in the nucleus of HDF. The local chromatin density was confirmed via transfection of H2B protein (H2B-GFP) labeled with GFP (green fluorescent protein). As time goes by after iExo treatment, the H2B-GFP distribution spread in a wider manner (FIG. 4E). Furthermore, expression of HP1α (heterochromatin protein 1α) in the nucleus and H3K27me3 as the inhibitory histone modification was reduced, and the activating histone modification H3K4me324 increased in the HDF as time elapsed after the iExo treatment (FIG. 4F). In particular, when the DNA methylation profile of the neurogenic-related gene was confirmed, the methylation was reduced (FIG. 4G). The result indicates that the reprosomes induce reprogramming of a cell into an NPC-like cell via several epigenetic regulators, as well as via activation of the MAPK signal transduction system and chromatin remodeling.

### Experiment Example 4. Analysis Experiment of in Vitro and In Vivo Differentiation Ability of rNPC

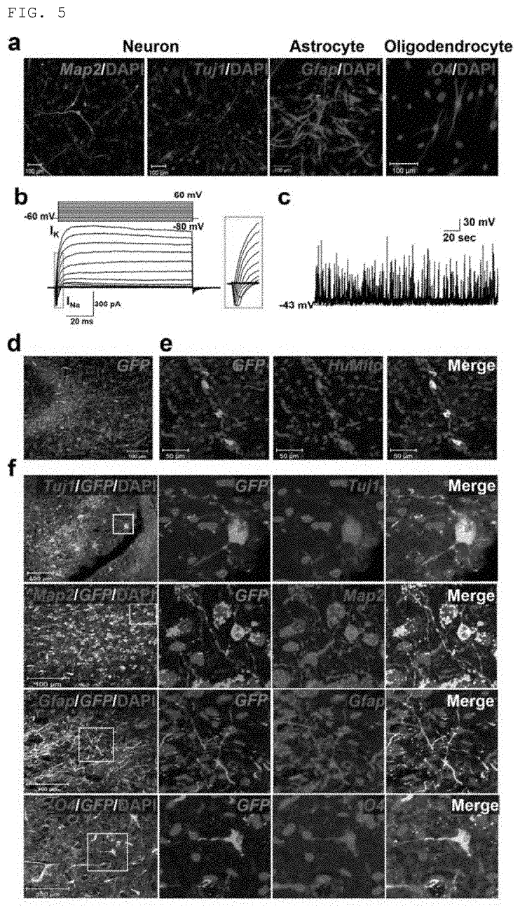

To evaluate the ability of rNPCs to differentiate into neural cells, the present inventors have examined whether rNPCs can be differentiated into a neural system such as neurons, astrocytes and oligodendrocytes. A 5-day old rNPC spheroid was cultured on a culturing plate with a gelatin coating, according to a recently published neural differentiation protocol. It was confirmed that Map2 and Tuj1 (neuron marker), Gfap (astrocyte marker), and O4 (oligodendrocyte marker) were expressed from the differentiated cells at 4 weeks after neural differentiation (FIG. 5A). From qRT-PCR analysis results of Map2, Tuj1, Gfap, S100b (astrocyte marker), Mbp (oligodendrocyte marker), and Oligol (oligodendrocyte marker) also confirmed that the rNPC was successfully differentiated into neural cells. Next, the functional properties of neurons derived from rNPCs were examined via whole-cell patch clamp recording. First, it was confirmed that the expression of genes associated with potassium ion channels (EAG1, Kv4.3, and Kv7.2) and sodium ion channels (Nav1.3, Nav1.6, and Nav1.7) increased greatly after the differentiation of rNPCs into neurons. At current clamps, in neurons derived from rNPCs, inward sodium and outward potassium currents was confirmed (FIG. 5B). Further, it was observed that sodium current was inhibited in neurons derived from the rNPC by a sodium ion channel blocker TTX (tetrodotoxin). Neurons derived from rNPCs induced active potentials in response to current clamps (FIG. 5C). The result shows that rNPC has the multipotency and is effectively differentiated into neurons, astrocytes and oligodendrocytes in vitro.

To determine whether rNPC could differentiate in vivo and in a multipotency manner, the present inventors have established a cell (GFP-HDF) in which a reporter is stably expressed into a G418 antibiotic, using HDF obtained by transfecting a GFP reporter expressed by a CMV (cytomegalovirus) promoter. The inventors transplanted an established 5-day old GFP-rNPC spheroids (approximately 1,500 clusters) to the brain (striata) of a normal Sprague Dawley rat (n=5) and identified the differentiation of these cells after 4 weeks. From the results, they confirmed GFP-positive cells in various parts of the brain. Many of them formed a long neurite (FIG. 5D). This may indicate that these cells have efficiently survived, migrated and been host-integrated. It was confirmed that GFP-positive cells were stained with human mitochondrial antibodies, and that these cells were GFP-rNPCs (FIG. 5E). Some of Gfap-positive astrocytes, Map2 and Tuj1-positive neurons, and 04-positive oligodendrocyte were GFP-positive (FIG. 5F). The result indicates that the rNPC induced from the transplanted HDF could have multipotency by which a cell may differentiate into three different cell types of the neural system in vivo within 5 days.

### Experiment Example 5. Analysis Experiment of Reprosomes with Adipocyte Inducing Ability

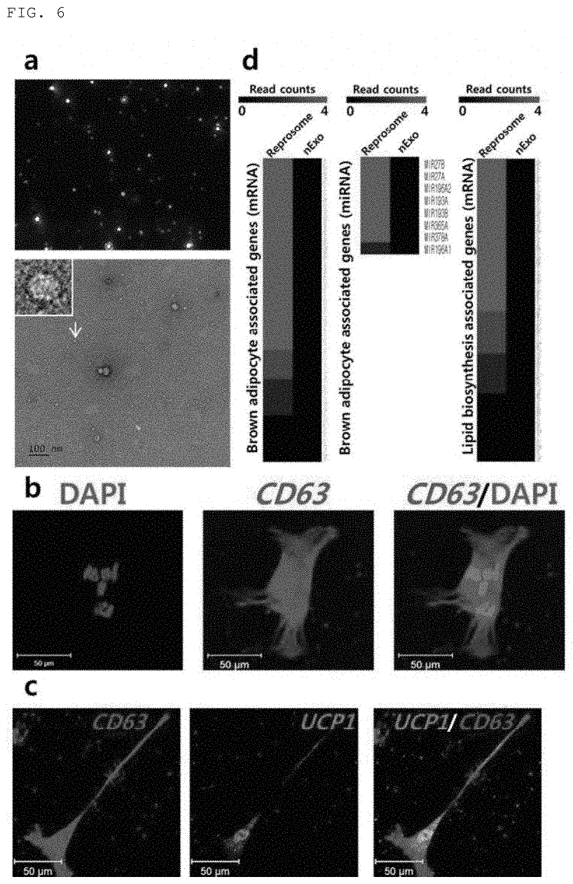

The present inventors analyzed UHDF cultured according to the method for inducing reprosomes having adipocyte inducing ability according to Example 2 via immunofluorescence staining for CD63 exosome marker (here, a contrast dye is nuclear dye DAPI). From the result of the analysis, it was confirmed that a large amount of exosome was produced (FIG. 6A). From the result of immunofluorescent staining of the marker and UCP1, a brown adipocyte marker, it was confirmed that the UCP1 was expressed in the secreted exosome (FIG. 6B). A morphology of the reprosome obtained by the method for producing reprosomes with adipocyte inducing ability according to Example 2 was analyzed using Nanosight and TEM (transmission electron microscopy). From the analysis results, a normal form of exosome was observed (FIG. 6C). From the result of RNA-Seq analysis for the reprosome, brown adipocyte-related mRNA and microRNA and lipid synthesis-related mRNA were significantly increased compared to the control (FIG. 6D). Comprehensively, it was confirmed that the reprosomes having the adipocyte inducing ability was successfully induced and obtained from the HDF via the method for producing reprosomes according to the Example 2.

### Experiment Example 6. rBA Analysis Experiment

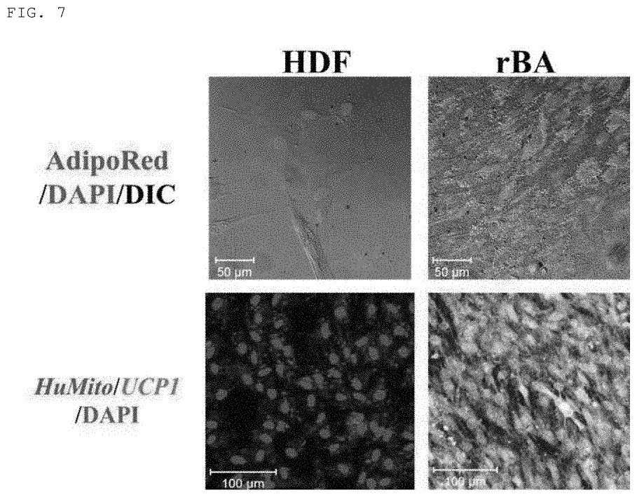

The present inventors stained the rBA produced according to the method for producing rBA according to Example 2 with AdipoRed which enables to confirm lipid droplets found in adipocyte in large quantities (here, the contrast dye is the nuclear dye DAPI). Compared to the control that showed AdipoRed-negative, the rBA showed a clear AdipoRed-positive (FIG. 7, top panel). The rBA was subjected to immunofluorescent staining with UCP1, a brown adipocyte marker (ehre, the contrast dye is the nuclear dye DAPI and a human mitochondrial antibody HuMito). Similarly, rBA was significantly UCP1-positive compared to the control that showed UCP1-negative (FIG. 7, bottom panel). The result shows that the HDF may be reprogrammed into the rBA via the reprosome produced according to the production method according to Example 2 and via the method for producing the rBA using the reprosome.

### Experiment Example 7. Analysis Experiment of Reprosome with Hepatocyte Inducing Ability

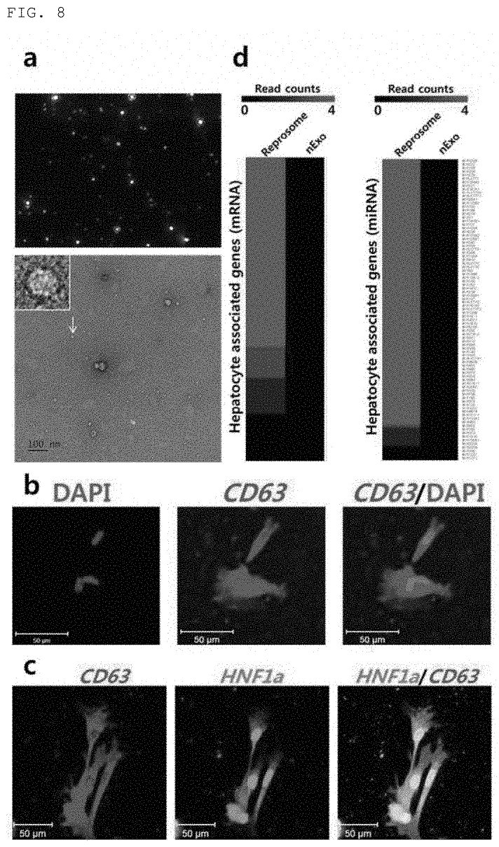

The present inventors analyzed a UHDF cultured according to the method for inducing reprosomes having hepatocyte inducing ability according to Example 3 via immunofluorescence staining for the CD63 exosome marker (here, the contrast dye is the nuclear dye DAPI). From the analysis result, it was confirmed that a large amount of exosome was produced (FIG. 8A). Immunofluorescent staining of the marker and hepatocyte marker HNF1a was performed, and the result showed that HNF1a was expressed in the secreted exosome (FIG. 8B). Nanosight and TEM (transmission electron microscopy) were used to analyze the morphology of reprosomes obtained according to the method for producing the reprosome having the hepatocyte inducing ability according to Example 3. From the analysis result, it was confirmed that a normal form of exosome was observed (FIG. 8C). Further, RNA-Seq analysis of the reprosome revealed that hepatocyte-related mRNA and microRNAs were significantly increased compared to the control (FIG. 8D). In general, it was confirmed that reprosomes having the adipocyte inducing ability were generated and secreted from the HDF via the method for producing reprosome according to Example 3.

### Experiment Example 8. rH Analysis Experiment

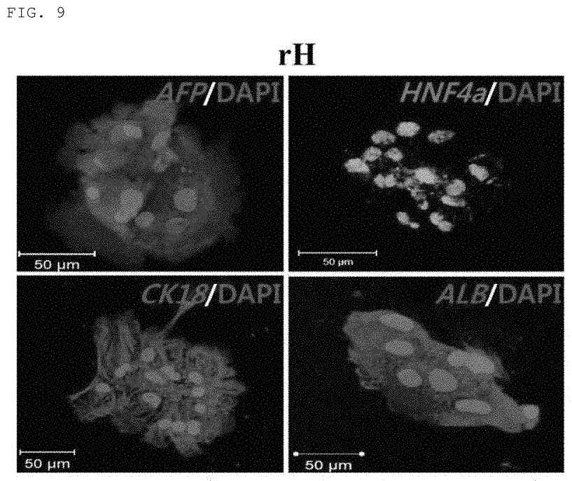

The present inventors performed immunofluorescent staining of rH produced according to the method for producing rH according to Example 3 using hepatocyte markers AFP, HNF4α, CK18, and ALB (here, the contrast dye is the nuclear dye DAPI). From the analysis result, it was confirmed that the rH showed a clear positive (FIG. 9). The result shows that HDF may be reprogrammed into the rH via the reprosome produced according to the method in the Example 3 and the method for producing the rH using the reprosome.

### Experiment Example 9. Analysis Experiment of Reprosome with Hair Regeneration Ability

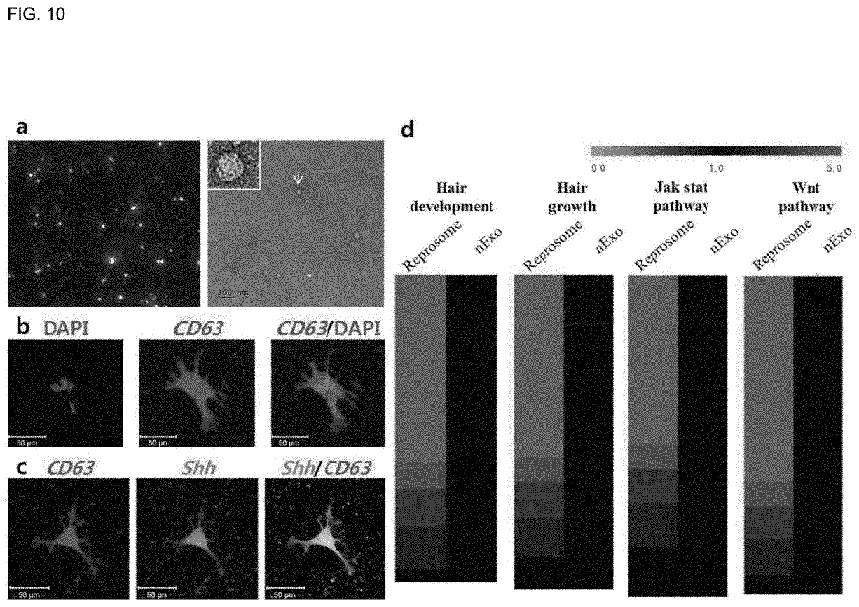

The present inventors analyzed the UHDF cultured according to the method for inducing reprosomes having the hair regeneration ability according to the Example 4 via the immunofluorescent staining for the CD63 exosome marker (here, the contrast dye is the nuclear dye DAPI). From the analysis result, it was confirmed that a large amount of exosome was produced (FIG. 10B). From the result of immunofluorescent staining of the marker and Shh (Sonic hedgehog), which is an important marker for hair regeneration, it was confirmed that Shh was expressed in the secreted exosome (FIG. 10C). A morphology of the reprosome obtained by the method for producing reprosome with hair regeneration ability according to the Example 4 was analyzed using Nanosight and TEM (transmission electron microscopy). From the analysis result, a normal form of exosome was observed (FIG. 10A). From the result of RNA-Seq analysis for the reprosome, hair regeneration-related hair tissue formation, hair growth stimulation, mRNA and microRNA of Jak-Stat signal transduction system and Wnt signal transduction system were significantly increased compared to the control (FIG. 10D). Thus, it may be confirmed that the method for producing reprosomes according to the Example 4 successfully induced the reprosome having the hair regeneration ability from the HDF.

### Experiment Example 10. Experiment of Hair Regeneration by Reprosome with Hair Regeneration Ability

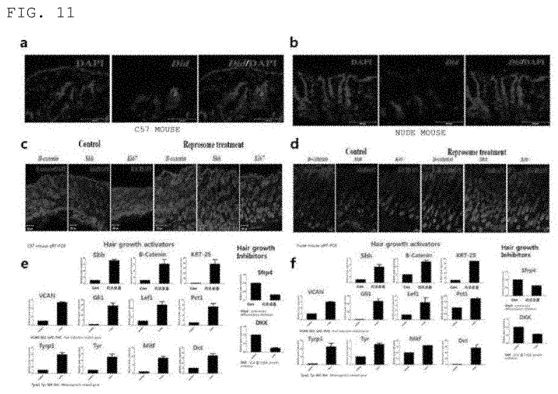

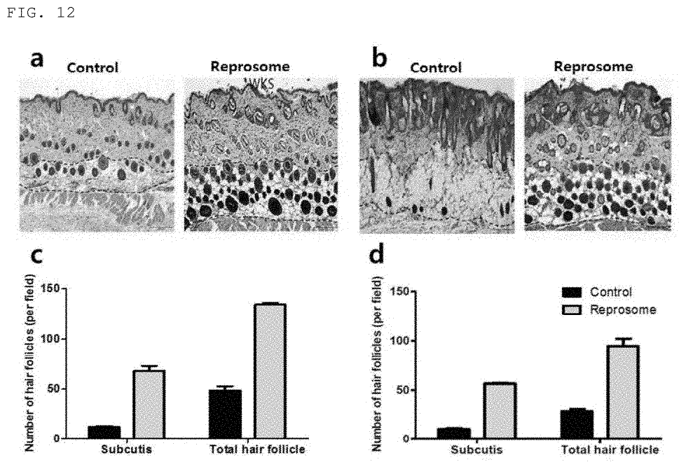

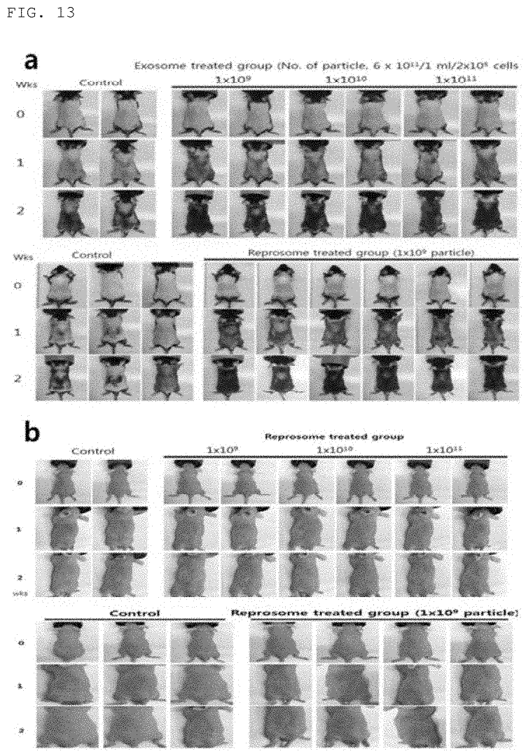

The present inventors stained the reprosomes obtained by the method for producing reprosomes having hair regeneration ability according to the Example 4 with a lipid marker DiD and applied to the epidermis of C57 mice, and the epidermis of nude mice as the control. It was confirmed that the reprosome penetrated into pores (FIGS. 11A and 11B, the contrast dye is the nuclear dye DAPI). The present inventors applied reprosomes diluted in a D-PBS culturing solution to the skin of the nude mice and the skin of the C57 mice in which dorsal side hairs were removed, at a concentration of 1×10⁹, 1×10¹⁰, and 1×10¹¹ cells/ml, respectively. The application effect was confirmed after one week and two weeks after the application. It was confirmed that in the case of the nude mice, hair was not observed with human naked eyes in a group not treated with reprosome, whereas in a group treated with reprosome, a large number of hair was grown from the first week. It was confirmed that in the C57 mice, a larger number of hair was grown longer compared to the control (FIGS. 13A and 13B). In the group treated with reprosome for 2 weeks, the hair was at least 3 times longer compared to the control. From the result of H&E staining, it was confirmed that a number of portions dyed dark purple representing hair follicles were observed in the skin treated with reprosome (FIGS. 12A and 12B). A much larger number of hair follicles were found in the entire skin layer, particularly in the subcutis compared to the control (FIGS. 12C and 12D). It was confirmed at the tissue level that the hair generation was promoted. Further, in the group treated with reprosome, an increase in the expression of β-catenin, Shh and Ki67, which are proteins involved in hair follicle cell regeneration, was confirmed via tissue immunofluorescent staining (FIGS. 10C and -10D). It was confirmed by the qRT-PCR test that an expression of mRNA of Shh, β-Catenin, KRT-25, VCAN, Gli1, Lef1, Pct1, Tyrp1, Tyr, Mitf, and DCT as the hair growth promoting factors increased, while an expression of mRAN of Sfrp4 and DKK as the hair growth inhibitory factor decreased (FIGS. 11E and 11F). Collectively, as predicted in the in vitro experiment according to the Experiment Example 9, the reprosome with the hair regeneration ability showed an excellent effect on hair regeneration in vivo.

### Experiment Example 11. Analysis Experiment of Reprosome with Tissue Regeneration Ability

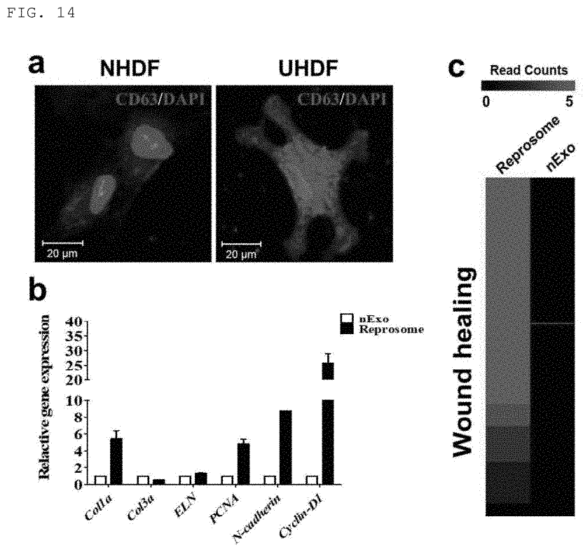

UHDF cultured according to the method for producing reprosomes with tissue regeneration ability according to Example 5 was analyzed by immunofluorescence staining for the CD63 exosome marker (here, the contrast dye is the nuclear dye DAPI). From the analysis, it was confirmed that a large amount of exosome was produced (FIG. 14A). The exosome (reprosome) isolated from the culturing solution for the cells was analyzed via qRT-PCR. From the analysis result, it was confirmed that the levels of expression of Collagen 1α (Collα), Collagen 3α (Col3α) and Elastin (ELN) as extracellular matrix-related genes associated with wound healing and the expression levels of proliferating cell nuclear antigen (PCNA), N-Cadherin and Cyclin-D1 as genes involved in cell proliferation were improved compared to the exosomes isolated from the control (FIG. 14B). Further, from the RNA-seq result, it was found that in the reprosome, more wound healing genes were expressed compared to nExo (FIG. 14C).

### Experiment Example 12. Analysis Experiment of Tissue Regeneration Effect by Reprosome with Tissue Regeneration Ability

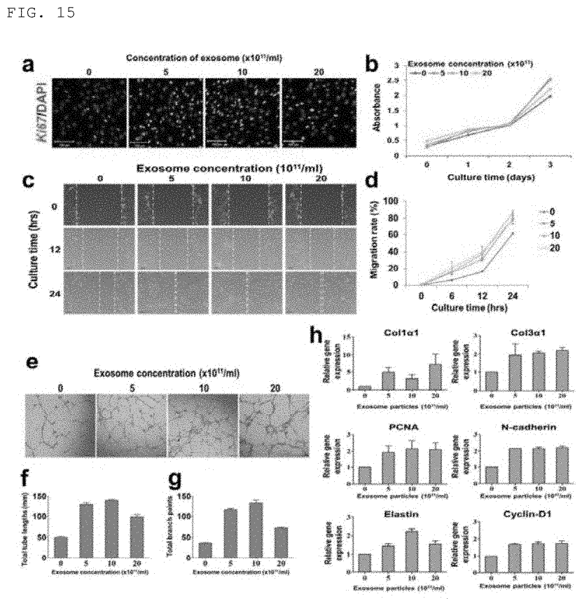

HDF was treated using the reprosome produced according to the method for producing reprosome with the tissue regeneration ability according to Example 5, and the effect based on the concentration of the reprosome was examined. First, the cell proliferation effect was analyzed via immunofluorescent staining for Ki67 as a marker associated with cell proliferation. From the analysis result, it was confirmed that the expression level of the marker was higher in an experiment group than the control. Especially, when HDF was treated using the reprosome with concentrations of 5×10¹¹ cells/ml and 10×10¹¹ cells/ml, the skin regeneration effect was excellent. It was further confirmed that the number of the cells was increased (FIGS. 15A and 15B). Next, the present inventors cultured the HDF so that the HDF completely filled the inside of the plate, drew a line with a 20 μl yellow tip, and separated the cells. Then, the cell was cultured together with the reprosome treatment to carry out the wound healing assay. From the assay result, it was shown that empty spaces were filled up relatively quickly in the reprosome treated group (FIGS. 15C and 15D), thus confirmed that a cell migration rate was high in the control.

Next, in order to analyze the angiogenic efficiency during the tissue regeneration process based on the concentration of reprosome produced according to the Example 5, endothelial cells HUVECs were treated with reprosomes with different concentrations. After 10 hours, tube formation was confirmed. From the analysis result, it was confirmed via naked eyes the formation of the tube may be promoted in the group treated with the reprosome compared to the control (FIG. 15E). The length of the tube was longer than that of the control and the number of crossing points was higher than that of the control (FIGS. 15F and 15G), especially high in the group treated with 5×10¹¹ cells/ml and 10×10¹¹ cells/ml of the reprosomes.

Finally, the expression of wound-healing-related genes in cells treated with reprosome was analyzed via qRT-PCR. From the analysis result, it was confirmed that the expression of the wound healing-related gene was higher in the experimental group than in the control (FIG. 15H).

Generally, the reprosomes produced according to the Example 5 may enhance cell proliferation, migration, and vascular development, thereby improving tissue wound healing and regeneration ability.

### Comparative Example 1. Control for Reprosome

Non-ultrasound-treated HDF (NHDF) was cultured in an hNMSC medium (StemPro®NSC SFM, Gibco) containing 2 mM GlutaMAX™-I Supplement (Gibco), 6 U/mL heparin (Sigma-Aldrich) and 200 μM ascorbic acid (Sigma-Aldrich) or fibroblast medium (DMEM (Gibco) containing 10% fetal bovine serum (Gibco) and 1% penicillin/streptomycin (Gibco). A process of separating exosome (nExo) from the culture medium used to culture the NHDF was the same as the reprosome separation method according to Example 1 above.

### Comparative Example 2. Control of Cells Induced by Reprosome

The present inventors seeded 1×10⁵ HDFs in a 35-mm petri dish and cultured the HDFs for one day. Subsequently, the existing medium was replaced with a medium containing exosome obtained according to Comparative Example 1 above. The HDF was cultured for 5 days. Here, the medium containing the exosome corresponded to each experiment group. Thus, the hNSC medium, adipocyte differentiation-inducing medium for a stem cell, and hepatocyte-culture medium were respectively used for controls for the rNPC, rBA and rH. The culture medium was replaced every two days.

The reprosomes in accordance with one embodiment of the present invention contain a variety of factors that can induce reprogramming of cells, particularly chromatin remodeling factors. Further, the reprosome has a phospholipid-based membrane structure, which facilitates penetration through the cell membrane and enhances the delivery efficiency of the substance. This allows induction of reprogramming into cells with desired functions with high efficiency. For example, the direct neural cell differentiation efficiency using existing human somatic cells is lower than 0.1%, while when using the reprosome, the direct cell differentiation efficiency reaches about 70%.

The method to produce the reprosomes according to one embodiment of the present invention may induce a large amount of reprosome secretion from somatic readily obtainable somatic cells as well as the stem cells and precursor cells, which are difficult to isolate and amplify, via a simple process including sonication. The yield of the thus induced exosome is higher than that of the conventional method. The amount and variety of the various factors incorporated in the exosome are also higher compared to the conventional method.

The reprogramming method of cells according to one embodiment of the present invention can safely induce reprogramming into cells with the desired function, without the introduction of chemicals or foreign transcription factors into the genome. Further, the reprogramming method can reprogram one type of cell into another in a relatively short period of time, without the need to go through several developmental stages, via simply adding the reprosome to the culture medium and culturing the cell.

The composition containing the reprosome according to one embodiment of the present invention may be administered to a body site to promote reprogramming of cells present in the body site, thereby promoting tissue regeneration of the treated body site.

The effect of the present invention is not limited to the above-defined effect. However, the effects of the present invention should be understood to cover all effects deduced from the description of the present invention or from the composition of the invention set forth in the claims.

The foregoing description of the present invention is for illustrative purposes only. Those of ordinary skill in the art to which the present invention belongs may understand that the present invention can be readily modified into other specific forms without departing from the spirit or essential characteristics of the present invention. It is to be understood, therefore, that the presently described embodiments are to be considered in all respects as illustrative and not restrictive. For example, each component described in a single manner may be implemented in a distributed manner. Likewise, components described in a distributed manner may also be implemented in a combined form.

The scope of the present invention is defined by the appended claims. It is intended that all changes and modifications derived from the meaning and scope of the claims and their equivalents be construed to be included in the scope of the present invention.

This application is based on Korean Patent Application No. 10-2018-0117261 filed Oct. 2, 2018, the disclosure of which is incorporated herein by reference in its entirety.

What is claimed is:

1. A reprosome capable of inducing cell reprogramming, wherein the reprosome comprises RNA of a gene involved in chromatin remodeling, wherein the gene includes a kinase gene on a mitogen-activated protein kinase (MAPK) signal transduction system, and a gene having histone modification activity.

2. The reprosome of claim 1, wherein the kinase gene is at least one selected from the group consisting of BRAF, MAP2K3, MAP3K10, MAP3K4, MAP3K5, MAP3K7, MAPK12, RPS6KA4(MSK2), TAOK1 and TAOK2.

3. The reprosome of claim 1, wherein the gene having the histone modification activity is at least one selected from the group consisting of ASH1L, CREBBP, DOT1L, EP300, GTF3C1, KAT2A, KAT6B, KDM1A, KDM3B, KDM6A, KMT2A, KMT2E, NCOA3, NSD1, SETD1A and SETD2.

4. The reprosome of claim 1, wherein a content of a small RNA in a total RNA in the reprosome is 40% or more, and a content of a microRNA (miRNA) in the small RNA is 40% or more.

5. A method for producing a reprosome capable of inducing cell reprogramming, the method comprising:
applying ultrasonic stimulation to a cell;
applying ultrasonic stimulation to a culture medium free of cells;
mixing the cell and the culture medium to form a mixture and culturing the cell in the mixture for a predetermined time; and
isolating the reprosome from the mixture.

6. The method of claim 5, wherein the cell includes a mammalian-derived fibroblast or a tissue cell in an organ.

7. The method of claim 5, wherein the culture medium is at least one selected from the group consisting of an embryonic stem cell medium, a neural stem cell medium, a cardiac stem cell medium, a dermal papilla cell medium, a mesenchymal stem cell medium, an osteogenic medium, a muscular formation medium, a hematopoietic stem cell medium, a neuron medium, an astrocyte medium, an oligodendrocyte medium, a hepatocyte medium, an adipocyte medium, a muscle cell medium, a vascular endothelial cell medium, a pancreatic beta cell medium, and a cardiac myocyte medium.

8. The method of claim 5, wherein the culture medium is any one selected from the group consisting of a neural stem cell medium, a dermal papilla cell medium, a hepatocyte medium, and an adipocyte medium.

9. The method of claim 5, wherein the ultrasonic stimulation applied to the cell is performed for 1 to 10 seconds at 10 to 30 kHz, and 0.5 to 3 W/cm².

10. The method of claim 5, wherein the ultrasonic stimulation applied to the culture medium is performed for 1 to 20 minutes at 10 to 30 kHz, and 1 to 20 W/cm².

11. The method of claim 5, wherein the culturing the cell in the mixture is carried out for 1 to 10 days.

12. The method of claim 5, wherein the isolating the reprosome from the mixture comprises:
after the culturing, centrifuging the mixture and obtaining a supernatant;
filtering the supernatant with a filter and obtaining a filtrate; and
concentrating the filtrate.

13. The method of claim 12, wherein the isolating the reprosome further comprises storing the supernatant at a temperature of 4° C. or below for 7 days to 1 month before filtering the supernatant with the filter.

14. The method of claim 12, wherein the isolated reprosome has a diameter of 50 to 200 nm.

15. A method for reprogramming a cell, the method comprising:
introducing the reprosome according to claim 1 into a first culture medium to form a mixture;
culturing a first cell in the mixture; and
obtaining a second cell after the culturing.

16. The method of claim 15, wherein the first cell comprises a mammalian-derived fibroblast or a tissue cell in an organ.

17. The method of claim 15, wherein the second cell has a differentiation ability equal to or lower than pluripotency.

18. The method of claim 15, wherein the second cell is any one selected from the group consisting of an embryonic stem cell, a neural stem cell, a cardiac stem cells, a dermal papilla cell, a mesenchymal stem cell, and a hematopoietic stem cell.

19. The method of claim 15, wherein the second cell is any one selected from the group consisting of a neural stem cell, a neuron, an astrocyte, an oligodendrocyte, a hepatocyte, an adipocyte, a hair follicle cell, a muscle cell, a vascular endothelial cell, a keratinocyte, a pancreatic beta cell, and a cardiac myocyte.

20. The method of claim 15, wherein the second cell is different from the first cell.

21. The method of claim 15, wherein the reprosome is introduced into the first culture medium at a concentration of 10⁷ to 10¹⁵ cells/ml.

22. The method of claim 15, wherein the first culture medium is the same as a culture medium used to produce the reprosome.

23. The method of claim 15, wherein the second cell is any one selected from the group consisting of a stem cell, a progenitor cell and precursor cell, and the culturing the first cell is performed for 1 to 6 days.

24. The method of claim 15, wherein the second cell is any one selected from the group consisting of a neuron, an astrocyte, an oligodendrocyte, a hepatocyte, an adipocyte, a hair follicle cell, a muscle cell, a vascular endothelial cell, a keratinocyte, a pancreatic beta cell, and a cardiac myocyte, and the culturing the first cell is performed for 10 to 60 days.

25. A composition comprising the reprosome of claim 1.
